(19) United States
(12) Patent Application Publication
(10) Pub. No.: US 2026/0248808 A1
(43) Pub. Date: Aug. 27, 2026

(54) Use Of Pyrithione Derivatives In The Treatment Of Colorectal Cancer

(71) Applicant: Xiamen University, Xiamen (CN)

(72) Inventors: Xiaonan WU, Xiamen (CN); Zhiping ZENG, Xiamen (CN); Cheng LEI, Xiamen (CN); Yutong HOU, Xiamen (CN)

(21) Appl. No.: 19/650,415

(22) Filed: Apr. 17, 2026

Related U.S. Application Data
(63) Continuation of application No. PCT/CN2025/121868, filed on Sep. 17, 2025.

(30) Foreign Application Priority Data
Feb. 24, 2025 (CN) 202510201812.0

(51) Int. Cl. A61K 31/555; A61P 35/00; C07F 1/08; C07F 3/06

(52) U.S. Cl. CPC A61K 31/555; A61P 35/00; C07F 1/08; C07F 3/06

(57) ABSTRACT

Pyrithione derivatives are used in the treatment of colorectal cancer, belonging to the field of biopharmaceutical technology. The affinity of zinc pyrithione (ZPT), copper pyrithione (CPT), and copper pyrithione monomers (CPT monomers) toward the epigenetic modification enzyme HSPBAP1 is verified, wherein the pyrithione derivatives can effectively target the HSPBAP1 protein. Moreover, in vitro migration and invasion assays are also performed on various colorectal cancer cell lines. It is found that ZPT, CPT, and CPT monomers can significantly inhibit the migration and invasion of various colorectal cancer cells in vitro, demonstrating that the pyrithione derivatives have broad-spectrum anti-metastatic capabilities against colorectal cancer.

### CROSS-REFERENCE TO RELATED APPLICATIONS

The present application is a continuation application of International Patent Application No: PCT/CN2025/121868, filed on Sep. 17, 2025, which claims the priority of the Chinese patent application for an invention under application No. “202510201812.0” and entitled “Use of pyrithione derivatives in the treatment of colorectal cancer”. The priority date is Feb. 24, 2025, and the applicant of the prior application is Xiamen University.

### REFERENCE TO SEQUENCE LISTING

A computer readable XML file entitled “GWPCTP20251002356-sequence listing”, which was created on Nov. 4, 2025 and has a file size of about 5,187 bytes, contains the sequence listing for this application and is hereby incorporated by reference in its entirety.

### TECHNICAL FIELD

The present application belongs to the field of biopharmaceutical technology, in particular relating to the use of pyrithione derivatives in the treatment of colorectal cancer.

### BACKGROUND

Colorectal cancer (CRC) is the third cancer category of high incidence worldwide with a mortality rate ranking second, making it the second lethal cancer across the world. According to the latest global cancer statistics report from the World Health Organization (WHO), it is suggested that new cases of CRC account for 10% of global cancer cases, ranking the third malignant tumor category of high incidence in the world after breast and lung cancers. CRC is a highly prevalent and lethal malignant tumor with a great unfulfilled demand for drugs, which is on the rise.

Activation of invasion and metastasis are hallmarks of cancer and the most prominent characteristics of malignant tumors. At present, metastasis remains the leading cause of cancer-associated death, and more than 90% of cancer patients die from metastasis. Despite the use of EGFR inhibitors and immunotherapies for patients with DNA mismatch repair deficiency, there is still a severe shortage of targeted drugs for most metastases of colorectal cancer. Metastasis is a cascade process that can be divided into three phases: dissemination, dormancy, and colonization. During this period, primary tumor cells acquire the ability to migrate aggressively, and become metastasis-initiating cells (MICs). MICs invade the blood or lymphatic vessels and disseminate through the circulatory system, becoming circulating tumor cells (CTCs). In the process of dissemination, most CTCs are eliminated physically, biochemically, or by oxidative or immune stress. Few CTCs invade distant organs through capillary extravasation, termed disseminated tumor cells (DTCs).

Most DTCs are eliminated by organ-specific or systemic immune defenses. Few DTCs can evade immune surveillance by entering a dormant state and then a proliferation state to form micrometastasis and macrometastasis. Each step in the cascade process of metastasis requires to metastasize cells thereby acquiring numerous traits, in which epigenetic regulation plays a vital role. By now many studies have revealed that the cascade process of metastasis involves multiple gene expression programs and mechanisms of epigenetic regulation, including DNA methylation, histone modifications, 3D chromatin organization, and non-coding RNAs, etc. They can respond to cellular stress by regulating gene expression without altering genomic DNA sequences, thereby reconstructing cell phenotypic traits.

Zinc pyrithione (ZPT) is a US Food and Drug Administration (FDA)-approved drug, as a topical antimicrobial agent for treating dandruff caused by Malassezia. It has a long history of safe and effective use and also has potential for treating prostate cancer, acute myeloid leukemia, oral cancer, lung cancer, liver cancer, ovarian cancer, melanoma, and triple-negative breast cancer to some extent. However, the effects of ZPT and derivatives thereof on colorectal cancer are still unknown.

### SUMMARY OF THE INVENTION

The present application provides use of a pyrithione derivative in the treatment of colorectal cancer, wherein the pyrithione derivative can target the epigenetic modification enzyme HSPBAP1 and inhibit the migration and invasion of colorectal cancer.

The present application provides use of a pyrithione derivative in the preparation of a medicament for the treatment of colorectal cancer, wherein the structure of the pyrithione derivative is shown in Formula I, and R in Formula I is selected from any of the following: Zn⁺, Cu⁺ and

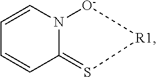

wherein R1 includes Zn²⁺ or Cu²⁺;

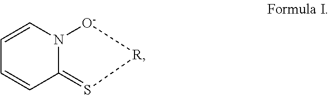

In one preferred embodiment of the present application, the therapeutic effect of the medicament for the treatment of colorectal cancer includes targeting the epigenetic modification enzyme HSPBAP1 with the pyrithione derivative to inhibit the migration and/or invasion of colorectal cancer.

In one preferred embodiment of the present application, the pyrithione derivative in the medicament for the treatment of colorectal cancer has an effective concentration of no less than 5 μM.

In one preferred embodiment of the present application, the pyrithione derivative includes any of the following:

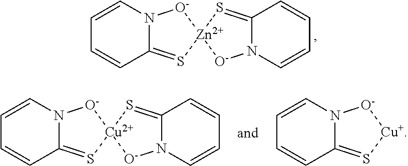

The present application further provides use of a pyrithione derivative in the preparation of an epigenetic modification enzyme HSPBAP1-targeted agent, wherein the structure of the pyrithione derivative is as shown in Formula I.

In one preferred embodiment of the present application, the pyrithione derivative in the epigenetic modification enzyme HSPBAP1-targeted agent has an effective concentration of no less than 5 μM.

In one preferred embodiment of the present application, the pyrithione derivative includes any of the following:

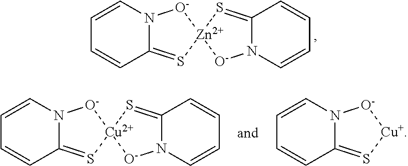

The present application further provides an epigenetic modification enzyme HSPBAP1-targeted agent, comprising a pyrithione derivative as an active ingredient and further a pharmaceutically acceptable adjuvant; the structure of the pyrithione derivative is shown in Formula I.

The present application further provides a medicament for inhibiting the migration and invasion of colorectal cancer, comprising a pyrithione derivative as an active ingredient and further a pharmaceutically acceptable adjuvant;
the structure of the pyrithione derivative is shown in Formula I.

In one preferred embodiment of the present application, the colorectal cancer includes colorectal cancer cell lines DLD1, HCT116, and SW480.

Beneficial effects: The present application provides a pyrithione derivative as shown in FIG. 1. In particular, it verifies the affinity of zinc pyrithione (ZPT), copper pyrithione (CPT), and copper pyrithione monomers (CPT monomers) toward the epigenetic modification enzyme HSPBAP1. Based on fluorescence titration assays, it is found that the in vitro dissociation constant KD value of ZPT toward the HSPBAP1 protein was 3.28 μM, indicating that ZPT can effectively target the HSPBAP1 protein. Moreover, in vitro migration and invasion assays are also performed on various colorectal cancer cell lines. It is found that 5 μM or 10 μM of ZPT, CPT, and CPT monomers could significantly inhibit the migration and invasion of various colorectal cancer cells in vitro, demonstrating that the pyrithione derivatives have broad-spectrum anti-metastatic capabilities against colorectal cancer.

### BRIEF DESCRIPTION OF THE DRAWINGS

FIG. 1 shows the formula of zinc pyrithione (ZPT).

FIG. 2 shows the formula of pyrithione (PT).

FIG. 3 shows the formula of copper pyrithione (CPT).

FIG. 4 shows the formula of a copper pyrithione monomer (CPT monomers).

FIG. 5A-FIG. 5D show the results of fluorescence titration assays of ZPT for the HSPBAP1 protein.

FIG. 6A-FIG. 6C show the results of ZPT inhibiting the migration and invasion of DLD1 cells.

FIG. 7A-FIG. 7C show the results of PT failing to inhibit the migration and invasion of DLD1 cells.

FIG. 8A-FIG. 8C show the results of CPT inhibiting the migration and invasion of DLD1 cells.

FIG. 9A-FIG. 9C show the results of CPT monomers inhibiting the migration and invasion of DLD1 cells.

FIG. 10A-FIG. 10C show the results of ZPT inhibiting the migration and invasion of HCT116 cells.

FIG. 11A-FIG. 11C show the results of ZPT inhibiting the migration and invasion of SW480 cells.

### DETAILED DESCRIPTION OF THE EMBODIMENTS

The present application provides use of a pyrithione derivative in the preparation of a medicament for the treatment of colorectal cancer, wherein the structure of the pyrithione derivative is shown in Formula I, and R in Formula I is selected from Zn⁺, Cu⁺ and

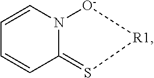

wherein R1 includes Zn²⁺ or Cu²⁺;

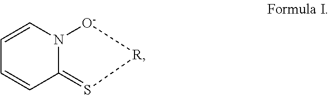

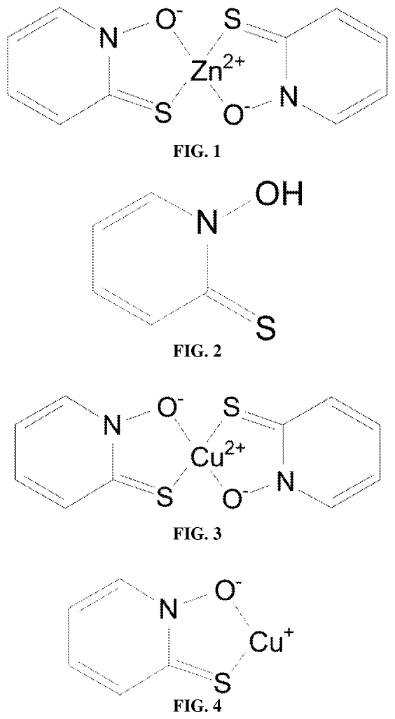

The structural formula of the pyrithione derivative in the present application is shown in FIG. 1, including zinc pyrithione (ZPT) as shown in FIG. 1, CAS No. 13463-41-7, having a molecular formula of C₁₀H₈N₂O₂S₂Zn and a molecular weight of 317.69; copper pyrithione (bis(1-hydroxy-1H-pyridine-2-thionato-o,s)copper) (CPT) as shown in FIG. 3, CAS No. 14915-37-8, having a molecular formula of C₁₀H₈CuN₂O₂S₂ and a molecular weight of 315.86; and a copper pyrithione monomer (CPT monomer) as shown in FIG. 4, which is a 1:1 complex formed by one copper atom and one pyrithione, CAS No. 154592-20-8, having a molecular formula of C₅H₅CuNOS and a molecular weight of 189.70. The present application also involves a comparative example using pyrithione (PT) as shown in FIG. 2, CAS No. 1121-30-8, having a molecular formula of C₅H₅NOS and a molecular weight of 127.16. The results show that, except for PT, all of ZPT, CPT, and CPT monomers can effectively inhibit the migration and invasion of colorectal cancer cells.

The present application does not specifically define the sources of the PT, CPT, CPT monomers, and ZPT. As in the example, all pyrithione derivatives were purchased from MedChemExpress (MCE), wherein ZPT is under the category No. HY-B0572, PT is under the category No. HY-B1747, CPT is under the category No. HY-W140346, and CPT monomers are under the category No. HY-W152501.

In one preferred embodiment of the present application, the pyrithione derivative in the medicament for the treatment of colorectal cancer has an effective concentration of no less than 5μ, e.g., 5μ M or 10 μM.

The present application further provides use of a pyrithione derivative in the preparation of an epigenetic modification enzyme HSPBAP1-targeted agent, wherein the structure of the pyrithione derivative is shown in Formula I.

In the example of the present application, fluorescence titration assays were performed to determine the dissociation constant of ZPT and its solvent DMSO for the HSPBAP1 protein. The results show that the KD value of ZPT for the HSPBAP1 protein was 3.28 μM, indicating that ZPT can strongly bind to HSPBAP1.

The present application further provides an epigenetic modification enzyme HSPBAP1-targeted agent comprising a pyrithione derivative as an active ingredient and further a pharmaceutically acceptable adjuvant; the structure of the pyrithione derivative is shown in Formula I.

The present application further provides a medicament for inhibiting the migration and invasion of colorectal cancer, the medicament comprising a pyrithione derivative as an active ingredient and further a pharmaceutically acceptable adjuvant;
the structure of the pyrithione derivative is as shown in Formula I.

In one preferred embodiment of the present application, the colorectal cancer includes colorectal cancer cell lines DLD1, HCT116 and SW480.

To further illustrate the present application, the following example is combined to describe the use of a pyrithione derivative in the treatment of colorectal cancer, provided by the present application, in detail, but they should not be construed as limiting the protection scope of the present application.

### Example 1

### I. Construction and Purification of a Prokaryotic Expression Vector for the Epigenetic Modification Enzyme HSPBAP1

1. On the website UCSC Genome Browser, the human HSPBAP1 gene was searched based on the GRCh38/hg38 genome; based on the mRNA transcript of the NCBI reference sequence NM_001320728.2, the full-length sequence of the HSPBAP1 protein (1-461 amino acids) encoded thereby was determined. Specifically, amino acids 1-260 of human HSPBAP1 represent the catalytic domain of histone demethylases. The DNA sequence encoding the protein is shown in SEQ ID NO: 1:

5′-ATGGCAGCAGGCTCCGAGGCGACCACTCCTGTGATCGTTGCGGCTGGGGCTGGAG
GGGAGGAAGGTGAACATGTCAAACCTTTTAAGCCAGAGAAAGCAAAAGAAATTA
TCATGTCTTTACAACAACCTGCAATCTTCTGTAACATGGTGTTTGATTGGCCAGCA
CGACACTGGAATGCTAAATACCTTTCGCAGGTCCTTCATGGCAAGCAGATACGATT
CAGAATGGGGATGAAAAGCATGAGCACAGTTCCTCAGTTTGAAACTACATGTAAT
TACGTAGAAGCTACACTCGAAGAGTTTCTGACCTGGAACTGTGACCAGTCTAGTAT
TTCTGGACCATTTAGAGATTATGACCATTCCAAGTTCTGGGCTTATGCTGACTATAA
ATATTTTGTCAGTCTATTTGAAGACAAGACAGATCTTTTCCAGGATGTGAAATGGT
CTGACTTCGGGTTTCCTGGAAGAAATGGACAGGAAAGTACATTGTGGATTGGCTC
CTTGGGAGCCCACACACCCTGTCATCTGGACTCCTATGGTTGTAACTTGGTATTCC
AGGTACAAGGAAGGAAACGATGGCATCTCTTTCCTCCTGAAGATACTCCTTTCCTT
TATCCAACTAGAATCCCTTATGAAGAATCTAGTGTGTTCAGTAAAATCAATGTTGTC
AATCCTGATTTAAAGCGTTTTCCTCAGTTCCGGAAAGCTCAAAGACATGCGGTTAC
ACTGAGCCCAGGACAGGAAGAGGATCACCTAGCCCGGGTAGAAGAGGCAATCACC-3′.

2. Herein the nucleotide sequence shown in SEQ ID NO: 1 was specifically amplified from a human cDNA library using PCR. The 5′ ends of the primers are attached to homologous sequences of the pGEX-4T1 vector multiple cloning site with BamH1 and Xho1 restriction sites, respectively. The specific PCR primer sequences are as follows:

F (SEQ ID NO: 2):
5′-GTTCCGCGTGGATCC GCAGCAGGCTCCGAGGCGACC-3′;
R (SEQ ID NO: 3):
5′-ATGCGGCCGCTCGAG GGTGATTGCCTCTTCTACCCG-3′.

PCR amplification system (25 μL): 5 μL of 5×PrimeSTAR Buffer (Mg²⁺ plus) (Takara), 2 μL of dNTP Mixture (Takara), 0.5 μL of Primer F (10 mM), 0.5 μL of Primer R (10 mM), 200 ng of cDNA, 0.5 μL of PrimeSTAR HS DNA Polymerase (2.5 U/μL) (Takara), and balance water.

PCR procedure: 98° C. for 5 minutes; 98° C. for 10 seconds, 56° C. for 30 seconds, 72° C. for 3 minutes, 30 cycles; 72° C. for 10 minutes; and stored at 4° C.

After completion of the PCR procedure, the PCR product was separated by agarose gel electrophoresis. The PCR product fragments, in a size of about 1.5 kb, were identified under UV light, excised, and recovered. Finally, the recovered DNA fragments were eluted with 20 μL of ultrapure water for the subsequent ligation reaction of molecular cloning.

3. The present application used the ligation independent cloning (LIC) to ligate the PCR product into the pGEX-4T1 vector, thereby generating the plasmid pGEX-4T1-HSPBAP1 (1-260) capable of expressing GST-tagged HSPBAP1 (1-260) truncated protein in large quantities in E. coli.

Digestion system: 1 μL of CutSmart® (NEB), 2 μg of pGEX-4T1 plasmid, 1 μL of BamHI (NEB), and 1 μL of Xhol I (NEB).

After the digestion reaction was fully completed for 7 hours, the DNA gel was run to find the enzyme-digested vector fragments under UV light, and then subjected to cutting and recovery. Finally, the recovered DNA fragments were eluted with 20 μL of ultrapure water and used as a vector for the subsequent ligation reaction.

Ligation system: 1 μL of 10× Exonuclease III Buffer (Takara), 3 μL of PCR product DNA, and 3 μL of digested vector DNA.

Ultrapure water was added to a total volume of 9 μL and stood on ice for 5 minutes. 1 μL of Exonuclease III (200 U/μL, Takara) was added and mixed evenly. After standing on ice for 1 hour, 0.5 μL of 0.5 M EDTA (pH 8.0) was added and mixed to terminate the reaction. The exonuclease activity was inactivated in a 60° C. water bath for 5 minutes. After standing on ice for 5 minutes, the LIC product was added to 100 μL of Turbo competent E. coli cells. The mixture stood on ice for 30 minutes and was heat-shocked in a 42° C. water bath for 45 seconds. The competent cells were evenly plated on an ampicillin antibiotics-containing bacterial plate medium via the dilution plating method, and stood at 37° C. overnight. The next day, single colonies formed after successful transformation were picked and shaken in liquid ampicillin antibiotics-containing bacterial medium. Plasmids were then extracted and sent for plasmid DNA sequencing, obtaining the correctly sequenced target plasmid pGEX-4T1-HSPBAP1 (1-260) with the correct insertion of the target fragments.

4. Next, the prokaryotic expression plasmid pGEX-4T1-HSPBAP1 (1-260) was transformed into the competent cells of E. coli BL21 Rosetta™ strain (Novagen). The Rosetta™ strain was supplemented with six rare tRNAs in E. coli, enabling universal translation. This facilitates translation of longer coding sequences by GST-HSPBAP1 (1-260), avoids the generation of truncated fragments due to premature translation termination caused by insufficient tRNAs, and promotes the expression of full-length fusion proteins. The GST-tagged HSPBAP1 (1-260) protein was purified according to the Glutathione Agarose (Thermo) product instructions. The specific methods for transformation and purification of bacteria are as follows:

### (1) Transformation

(a) Transfer 0.5 μL of plasmid pGEX-4T1-HSPBAP1 (1-260) into 50 μL of Rosetta and place in an ice bath for 30 minutes;

(b) Heat shock in a 42° C. water bath for 60 seconds;

(c) Quickly place in an ice bath for 5 minutes;

(d) Streak on a preheated ampicillin-resistant LB solid medium plate;

(e) Place in a 37° C. incubator upside down for incubation overnight.

### (2) Induction

(a) Small-scale culture. Add 20 mL of the LB liquid medium to a 50 mL centrifuge tube and then add ampicillin having a final concentration of 1 mM. Collect a single clone and transfer the single clone to a 50-mL centrifuge tube. Incubate the culture with shaking in a shaker at 220 rpm and 37° C. for 8 hours.

(b) Expand the culture. Add 1 L of LB liquid medium to a 2 L Erlenmeyer flask and add ampicillin having a final concentration of 1 mM. Transfer 20 mL of the bacterial solution to the 1 L of LB liquid medium. Continue to incubate with shaking in a shaker at 220 rpm and 37° C. for 3-4 hours until the OD value is 0.7-0.8. Then, add IPTG having a final concentration of 1 mM and incubate overnight (12 hours) with shaking in a shaker at 100 rpm and 25° C.

### (3) Purification

(a) Bacteria harvest. Centrifuge the bacterial solution at 4500 rpm for 15 minutes and discard the supernatant.

(b) Resuspension and lysis. Resuspend the bacteria in 40 mL of PBS buffer. Dissolve 40 mg of lysozyme (1 mg/mL) in 2 mL of PBS+1% Triton-X100 buffer and add to the resuspended bacteria solution. Place the bacteria solution in an ice bath for 30 minutes with gently shaking three times. Then, add 10% Triton-X100 to a final concentration of 1% Triton-X100. Ultrasonicate the bacterial solution for 15 minutes using an ultrasonic cell disruptor at 10 s ON and 15 s OFF and a power ratio of 80%.

(c) Incubation by centrifugation. After sonication, centrifuge the bacterial suspension at 13,000-15,000 rpm and 4° C. for 30 minutes, and transfer the supernatant to a new 50 mL centrifuge tube. Centrifuge 500 μL of 50% glutathione agarose beads at 700 G and 4° C. for 2 minutes, remove the supernatant, and resuspend in 1 mL of PBS+1% Triton-X100 buffer and mix them. Centrifuge at 700 G and 4° C. for 2 minutes, and remove the supernatant. Repeat three times, transfer to a 50 mL centrifuge tube, and incubate at 4° C. with rotation for 4 hours.

(d) Elution by centrifugation. After incubation, centrifuge at 1000 G and 4° C. for 5 minutes. Remove the supernatant while retaining the agarose beads. Resuspend and mix in 1 mL of PBS+1% Triton-X100 buffer, transfer to a 1.5 mL EP tube, and centrifuge at 700 G and 4° C. for 2 minutes. Remove the supernatant. Repeat four times. Resuspend the pellets and mix again in 1 mL of PBS buffer, centrifuge the suspension at 700 G and 4° C. for 2 minutes, and remove the supernatant. Repeat two more times. Add 1 mL of 50 mM Tris-HCl (pH 8.0)/10 mM reduced glutathione elution to incubate for 10 minutes. Transfer the mixture to an elution column and elute the target protein into a 1.5 mL EP tube.

### II. In Vitro Affinity Assay of ZPT Toward the HSPBAP1 Enzyme

The in vitro affinity of ZPT toward the HSPBAP1 protein was determined using fluorescence quenching. To be specified:

GST-tagged HSPBAP1 (1-260) protein that was eluted and purified in vitro, and the buffer 50 mM Tris-HCl (pH 8.0)/10 mM reduced glutathione solution were used for fluorescence quenching assay. The HSPBAP1 protein was incubated with ZPT having an increased concentration gradient at 25° C. and then the detection was performed using the Varian Cary Eclipse fluorescence spectrophotometer. The dissociation constant was determined by quantitative fitting of the spectrum of protein fluorescence quenching caused by ZPT.

Using a 300 μL quartz cuvette, 200 μL of the HSPBAP1 (1-260) protein solution (protein concentration of 5 μM) was added each time. The compound was diluted in DMSO to prepare a 10 mM stock solution. Taking ZPT as an example, which has a molecular weight of 317.69 g/mol, 1 mg of ZPT was dissolved in 314.4 μL of DMSO to prepare a 10 mM ZPT stock solution. Subsequently, the 10 mM stock solution was diluted with 50 mM Tris-HCl (pH 8.0) to a 200 μM solution. After the protein and compound solutions were equilibrated at room temperature for 30 minutes, the fluorescence spectrum of the protein solution was measured. Fluorescence quantification was performed using the Varian Cary Eclipse fluorescence spectrophotometer in scan mode, with an excitation wavelength of 280 nm and a slit width of 10 nm. The emission spectrum was scanned from 290 to 450 nm. Each sample was determined three times. Titration was then initiated by adding 2 μL of the compound solution to 200 μL of the protein solution (diluted by about 100-fold). After the completion of the first addition, the compound in the system had a concentration of 2 μM. The fluorescence spectrum was determined after mixing by pipetting. Later, the compound was added dropwise to the protein solution in the same manner, gradually generating a titration system of the compound (0, 2, 4, 6, 8, 10, 12, 14, 16, 18, 20, 22, 24, 26, 28, and 30 μM, in total of 15 times). Using the Graphpad Prism software, the area under the curve (AUC) of the protein-compound solution from 290 to 450 nm was obtained. The changed AUC relative to the control sample at each titration concentration was calculated, i.e., ΔAUC. By nonlinear fitting under the one site-total option, the dissociation constant of the compound for the protein was finally determined.

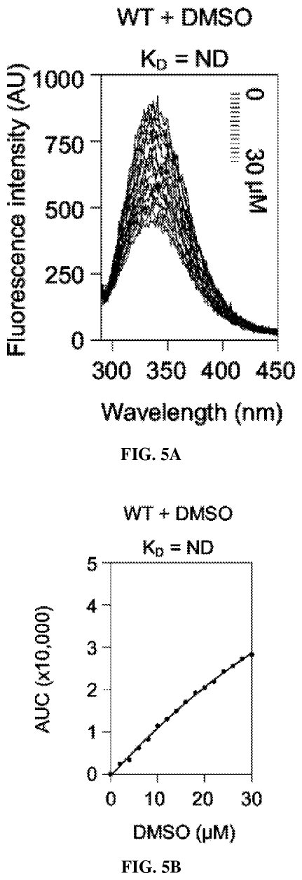

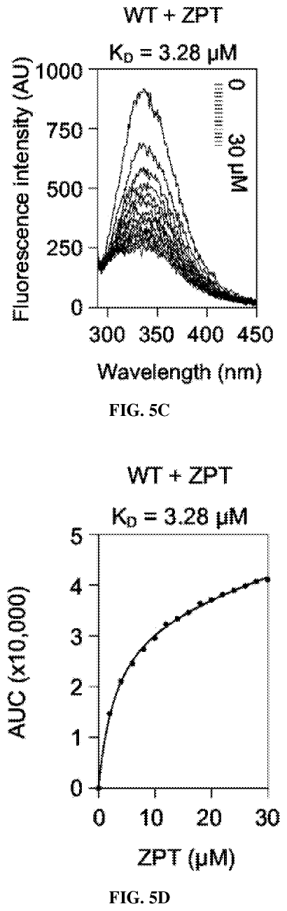

The dissociation constants of ZPT and its solvent DMSO for the HSPBAP1 protein are shown in FIGS. 5A-5D. The KD value of ZPT for the HSPBAP1 protein was 3.28 μM, indicating that ZPT can strongly bind to HSPBAP1.

### III. Cell Migration Assay.

Three common colorectal cancer cell lines, DLD1, HCT116, and SW480, were cultured in a 5% CO₂ incubator at 37° C. To be specified:
DLD1 cells were cultured in RPMI-1640 medium (BioInd, 01-100-1ACS) containing 10% fetal bovine serum (FBS) (BioInd, 04-001-1A);
HCT116 and SW480 cells were cultured in DMEM medium (BioInd, 01-052-1ACS) containing 10% FBS (BioInd, 04-001-1A).

Cells were digested with 0.25% trypsin (BioInd, 03-050-1A) and then resuspended in the corresponding medium containing 0.5% FBS. After counting, cells were seeded in a volume of 200 μL into cell culture inserts (NEST, 725301) having a pore size of 8 μm. DLD1 cells were seeded at 50,000 cells/insert, HCT116 cells at 100,000 cells/insert, and SW480 cells at 100,000 cells/insert. For the agent-feeding treatment with ZPT and derivatives thereof, ZPT and derivatives thereof were diluted to a final concentration of 5 or 10 μM and added to the upper compartment of the cell culture. The inserts for experiment were then placed in the center of a 24-well plate. 600 μL of the corresponding medium containing 20% FBS was added to the corresponding wells of the 24-well plate in the lower compartment. PBS was added to the edge wells of the 24-well cell culture plate to prevent evaporation of the medium from the laboratory inserts due to prolonged incubation. After a certain incubation period, cells were sampled, and the number of cells that passed through the pore size of the inserts was determined. DLD1 cells were cultured for 24 hours, HCT116 cells for 60 hours, and SW480 cells for 48 hours.

After completion of the above incubation, the cell culture plate was removed, and the cells in the upper compartment were gently removed with a cotton swab. Afterwards, the medium was discarded, and 200 and 500 μL of PBS were added to the upper and lower compartments for washing, respectively. Then, 200 and 500 μL of 4% paraformaldehyde were added, respectively. After fixing at room temperature for 10 minutes, the paraformaldehyde solution was discarded. 200 and 500 μL of 0.15% crystal violet solution were added to the upper and lower compartments, respectively, to stain at room temperature for 20 minutes. After staining was completed, the inserts were rinsed three times with tap water, and the wells and plate were placed in a well-ventilated area for air drying. Finally, three fields of view of each sample were examined using an inverted microscope at a 10× objective, and the number of cells migrating (or invading) across the membrane was counted using the ImageJ software.

### IV. Cell Invasion Assay.

A basement membrane matrix (BD, 356234) was placed in a 4° C. refrigerator overnight. The next day, the basement membrane matrix was diluted with the corresponding medium at a ratio of 1:10. 50 μL of the diluted basement membrane matrix was added to each insert and stood in a 37° C. incubator for 1 hour. Subsequently, cell seeding, agent feeding, staining, and quantification were performed as described above for cell migration assays.

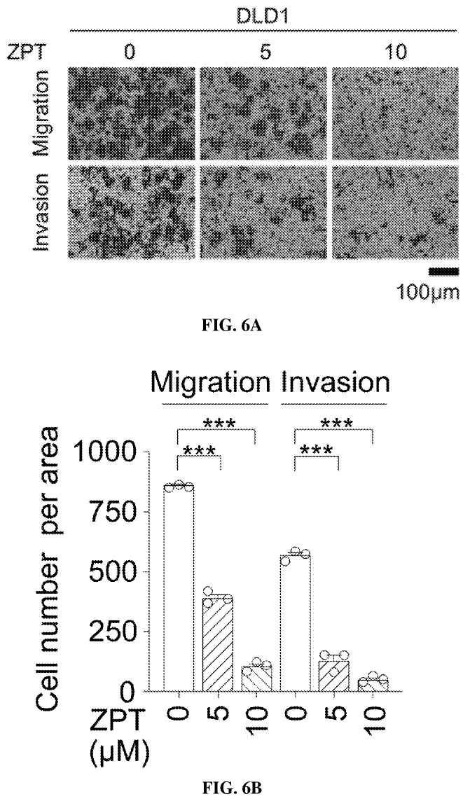

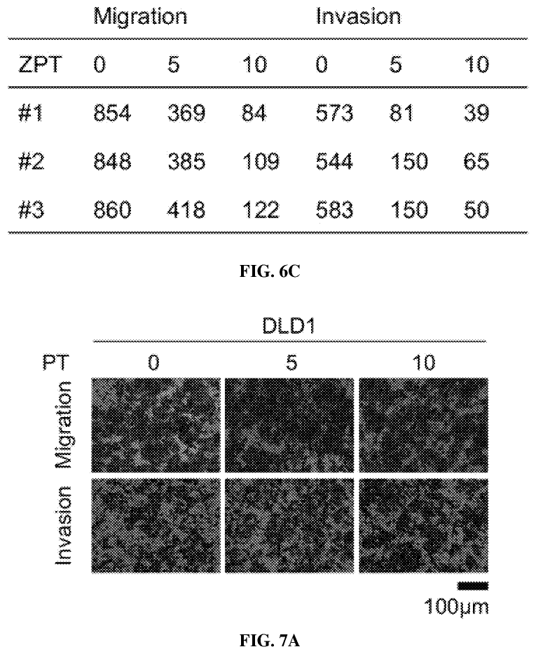

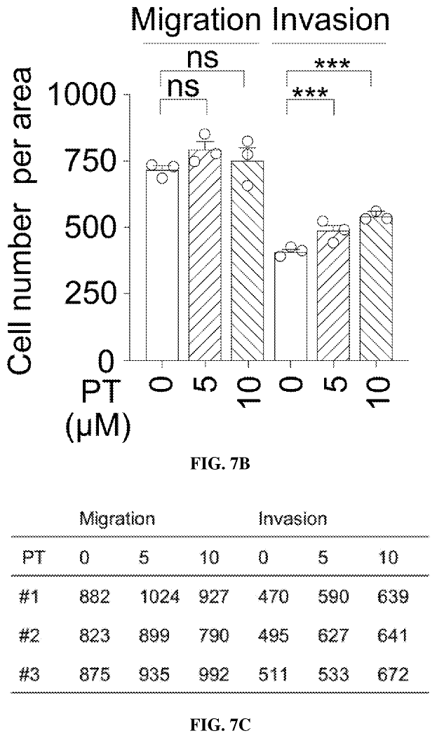

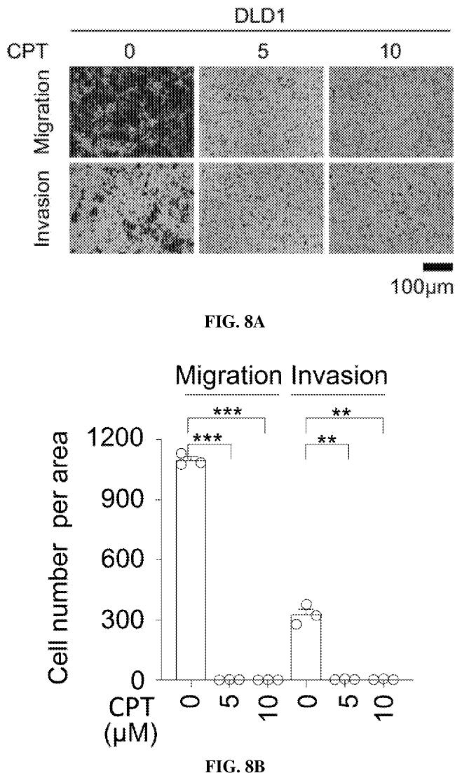

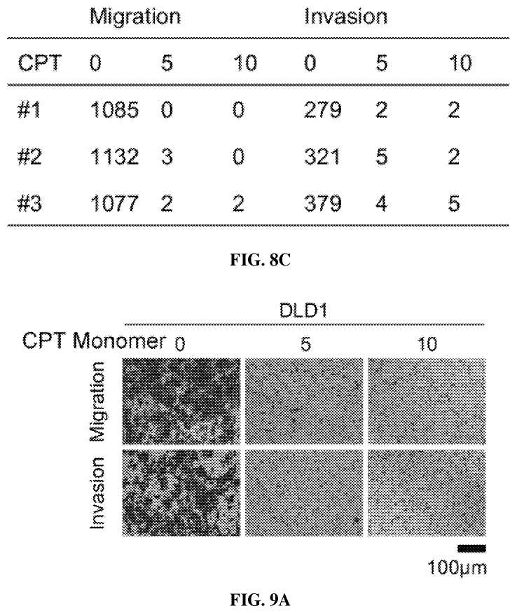

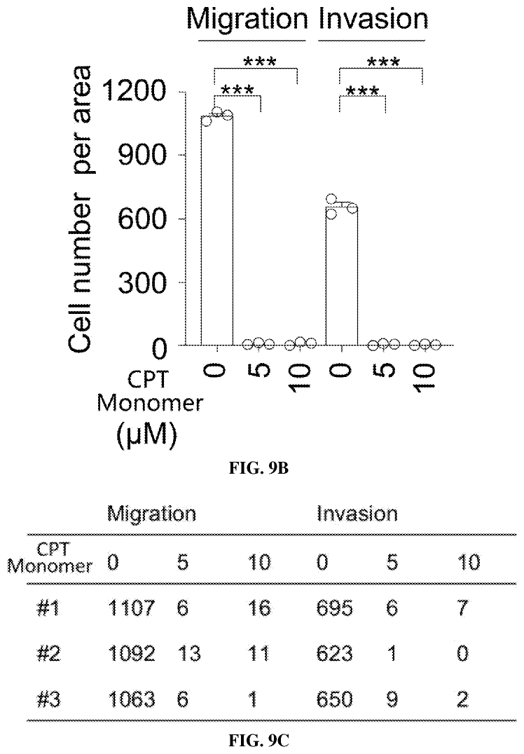

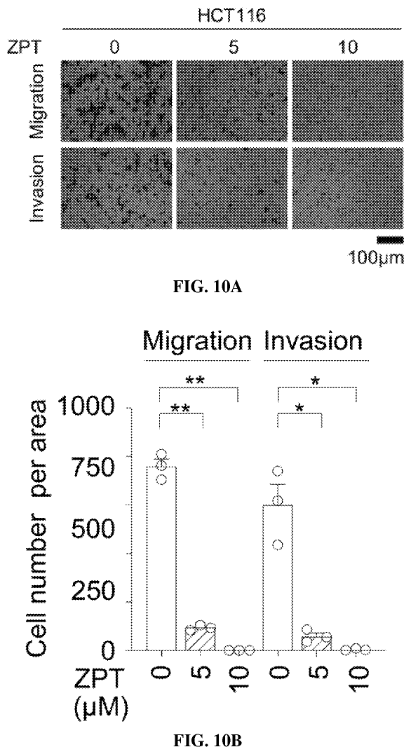

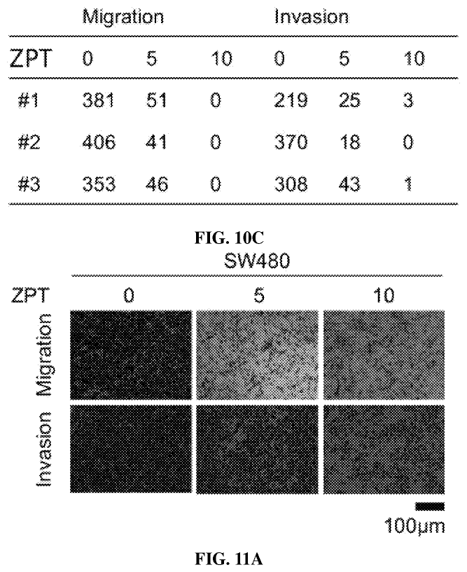

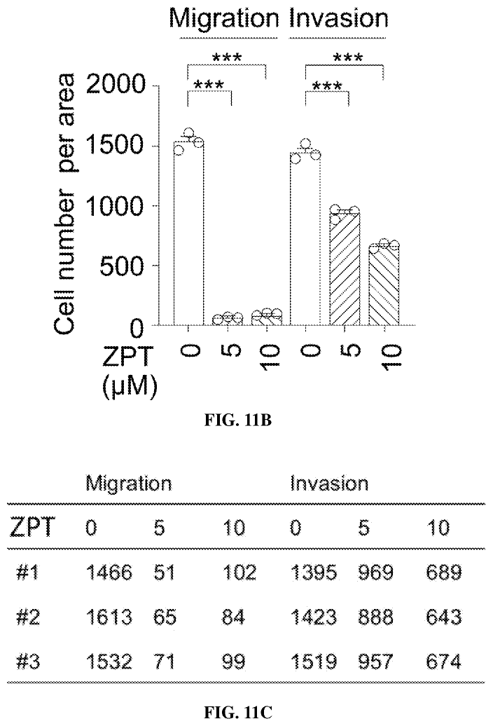

As shown in FIGS. 6A-6C, treatment of DLD1 cells with 5 or 10 μM ZPT for 24 hours significantly inhibited the migration and invasion of DLD1 cells. As shown in FIGS. 7A-7C, treatment of DLD1 cells with 5 or 10 μM PT for 24 hours failed to inhibit the migration and invasion of DLD1 cells. As shown in FIGS. 8A-8C, treatment of DLD1 cells with 5 or 10 μM CPT for 24 hours significantly inhibited the migration and invasion of DLD1 cells. As shown in FIGS. 9A-9C, treatment of DLD1 cells with 5 or 10 μM CPT monomers for 24 hours significantly inhibited the migration and invasion of DLD1 cells. As shown in FIGS. 10A-10C, treatment of HCT116 cells with 5 or 10 μM ZPT for 60 hours significantly inhibited the migration and invasion of HCT116 cells. As shown in FIGS. 11A-11C, treatment of SW480 cells with 5 or 10 μM ZPT for 48 hours significantly inhibited the migration and invasion of SW480 cells.

In summary, ZPT can inhibit the migration and invasion of DLD1 cells, HCT116 cells, and SW480 cells; CPT and CPT monomers can inhibit the migration and invasion of DLD1 cells; and PT without metal ion coordination cannot inhibit the migration and invasion of DLD1 cells.

Although the above example has described the present application in detail, it is only a part of, rather than all of, the embodiments of the present application. Those skilled in the art would also get access to other examples based on this Example without inventive labor, and these examples all belong to the protection scope of the present application.

What is claimed is:

1. A method for treating colorectal cancer, comprising administering a medicament comprising a pyrithione derivative to a subject in need thereof, wherein the pyrithione derivative has a structure shown in Formula I, and wherein R in Formula I is selected from Zn⁺, Cu⁺ and 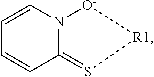
wherein R1 is Zn²⁺ or Cu²⁺;

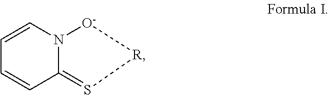

2. The method according to claim 1, wherein the method comprises targeting an epigenetic modification enzyme HSPBAP1 with the pyrithione derivative to inhibit the migration and/or invasion of colorectal cancer.

3. The method according to claim 2, wherein the colorectal cancer cells include DLD1 cells, HCT116 cells, and SW480 cells.

4. The method according to claim 1, wherein the pyrithione derivative in the medicament for the treatment of colorectal cancer has an effective concentration of no less than 5 μM.

5. The method according to claim 4, wherein the pyrithione derivative includes any of the following compounds:

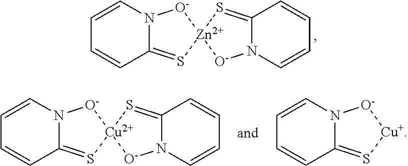

6. The method according to claim 5, wherein the compound 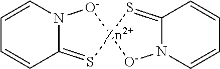
in the pyrithione derivative inhibits the migration and invasion of DLD1 cells, HCT116 cells and SW480 cells;
and the compounds 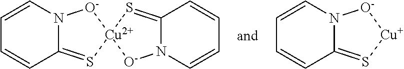
in the pyrithione derivative inhibits the migration and invasion of DLD1 cells.

7. A medicament for treating colorectal cancer, wherein the medicament comprises a pyrithione derivative as an active ingredient and further a pharmaceutically acceptable adjuvant;
wherein the pyrithione derivative has a structure shown in Formula I, and wherein R in Formula I is selected from Zn⁺, Cu⁺ and 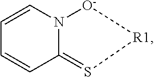
wherein R1 is Zn²⁺ or Cu²⁺;

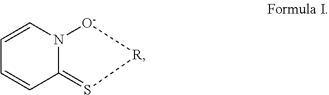

8. An epigenetic modification enzyme HSPBAP1-targeted agent comprising a pyrithione derivative, wherein the pyrithione derivative has a structure shown in Formula I, and wherein R in Formula I is selected from Zn⁺, Cu⁺ or 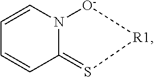
wherein R1 is Zn²⁺ or Cu²⁺;

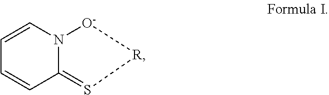

9. The epigenetic modification enzyme HSPBAP1-targeted agent according to claim 8, wherein the pyrithione derivative in the epigenetic modification enzyme HSPBAP1-targeted agent has an effective concentration of no less than 5 μM.

10. The epigenetic modification enzyme HSPBAP1-targeted agent according to claim 9, wherein the pyrithione derivative includes any of the following compounds:

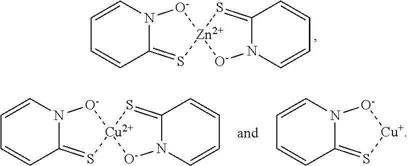

11. The epigenetic modification enzyme HSPBAP1-targeted agent according to claim 8, wherein the epigenetic modification enzyme HSPBAP1-targeted agent further comprises a pharmaceutically acceptable adjuvant.

12. A medicament for inhibiting according to claim 7, wherein the medicament inhibits the migration and invasion of colorectal cancer.

13. The medicament according to claim 12, wherein the colorectal cancer includes colorectal cancer cell lines DLD1, HCT116, and SW480.


SEQUENCE LISTING (3 sequences)

SEQ ID NO: 1 - DNA, 780 bp, Homo sapiens
  source 1..780: mol_type = other DNA; note = DNA sequence encoding the protein HSPBAP1
atggcagcaggctccgaggcgaccactcctgtgatcgttgcggctggggctggaggggaggaaggtgaacatgtcaaaccttttaagccagagaaagcaaaagaaattatcatgtctttacaacaacctgcaatcttctgtaacatggtgtttgattggccagcacgacactggaatgctaaatacctttcgcaggtccttcatggcaagcagatacgattcagaatggggatgaaaagcatgagcacagttcctcagtttgaaactacatgtaattacgtagaagctacactcgaagagtttctgacctggaactgtgaccagtctagtatttctggaccatttagagattatgaccattccaagttctgggcttatgctgactataaatattttgtcagtctatttgaagacaagacagatcttttccaggatgtgaaatggtctgacttcgggtttcctggaagaaatggacaggaaagtacattgtggattggctccttgggagcccacacaccctgtcatctggactcctatggttgtaacttggtattccaggtacaaggaaggaaacgatggcatctctttcctcctgaagatactcctttcctttatccaactagaatcccttatgaagaatctagtgtgttcagtaaaatcaatgttgtcaatcctgatttaaagcgttttcctcagttccggaaagctcaaagacatgcggttacactgagcccaggacaggaagaggatcacctagcccgggtagaagaggcaatcacc

SEQ ID NO: 2 - DNA, 36 bp, synthetic construct
  source 1..36: mol_type = other DNA; note = forward primer for PCR amplification of SEQ ID NO: 1
gttccgcgtggatccgcagcaggctccgaggcgacc

SEQ ID NO: 3 - DNA, 36 bp, synthetic construct
  source 1..36: mol_type = other DNA; note = Reverse primer for PCR amplification of SEQ ID NO: 1
atgcggccgctcgagggtgattgcctcttctacccg